(19) United States
(12) Patent Application Publication
(10) Pub. No.: US 2006/0177415 A1
(43) Pub. Date: Aug. 10, 2006

(54) Once a day formulation for phosphate binders

(76) Inventor: Steven K. Burke, Sudbury, MA (US)

Correspondence Address: GENZYME CORPORATION;LEGAL DEPARTMENT, 15 PLEASANT ST CONNECTOR, FRAMINGHAM, MA, 01701-9322, US

(21) Appl. No.: 11/262,502

(22) Filed: Oct. 27, 2005

Related U.S. Application Data
(60) Provisional application No. 60/623,985, filed on Nov. 1, 2004.

(51) Int. Cl. A61K 31/765

(52) U.S. Cl. 424/78.27; 424/78.3

(57) ABSTRACT

A method for reducing serum phosphate in a subject in need thereof comprising administering once per day to said subject a phosphate binder, wherein the phosphate binder has a phosphate binding capacity of at least 52 mmole.

### RELATED APPLICATION

This application claims the benefit of U.S. Provisional Application No. 60/623,985, filed on Nov. 1, 2004. The entire teachings of the above application are incorporated herein by reference.

### BACKGROUND OF THE INVENTION

Hyperphosphatemia frequently accompanies diseases associated with inadequate renal function, hyperparathyroidism, and certain other medical conditions. Hyperphosphatemia is typically defined for humans as a serum phosphate level of greater than about 4.5 mg/dL. The condition, especially if present over extended periods of time, leads to severe abnormalities in calcium and phosphorus metabolism and can be manifested by aberrant calcification in joints, lungs and eyes.

The oral administration of certain phosphate binders, to bind intestinal phosphate and prevent absorption, has also been suggested. Typical phosphate binders include calcium and aluminum salts. More recently, lanthanum and iron salts have been used as phosphate binders.

Anion exchange polymers, such as aliphatic amine polymers, have also been used in the treatment of hyperphosphatemia. These polymers provide an effective treatment for decreasing the serum level of phosphate, without concomitantly increasing the absorption of any clinically undesirable materials.

Phosphate binders are more effective at binding dietary phosphate than endogenous phosphate. Therefore, phosphate binders are currently administered with meals, to bind dietary phosphate before it is absorbed by the body and thus optimize the phosphate binding efficiency. Phosphate binding efficiency is believed to be greatly reduced when the binder is administered while fasting or more than two hours before or after a meal. This is demonstrated in Schiller et al. (N. Engl. J. Med. 1989: (320) 1110-1113) by a marked decrease in phosphate binding efficiency when the binder was administered to a subject two hours after a meal.

The need to take a phosphate binder with each meal places a burden on a patient and leads to problems with patient compliance and thus the effectiveness of the therapy. It is inconvenient for patients to take a medication at least two or three times a day, and patients tend not to adhere to such a strict regimen. Such a regimen also leads to further inconveniences such as the patient having to carry a supply of medication with them when eating out. A therapy with a reduced dosage frequency would be much more desirable in order to improve patient compliance and the efficiency of the therapy.

### SUMMARY OF THE INVENTION

It has now been found that a once-per-day phosphate binder formulation is substantially equivalent to a standard formulation requiring three times per day dosing for controlling serum phosphate. As shown in Example 1, after an eight week study, patients receiving sevelamer once per day had a serum phosphate level of 5.0±0.3 mg/dL which is statistically equivalent to patients receiving sevelamer three times a day who had a serum phosphate level of 4.6±0.3 mg/dL.

In one embodiment, the present invention is a method for reducing serum phosphate in a subject in need thereof comprising administering once per day to said subject a phosphate binder, wherein the phosphate binder has a phosphate binding capacity of at least 52 mmole. In a particular embodiment, the phosphate binder is an aliphatic amine polymer, preferably sevelamer. In another particular embodiment the phosphate binder is a pharmaceutically acceptable lanthanum salt.

In other embodiments, the present invention is a method for reducing serum phosphate in a subject in need thereof, comprising administering once per day to said subject at least 2 g of an aliphatic amine polymer, at least 2 g of sevelamer, or at least 0.5 g of a lanthanum salt.

In another embodiment the present invention is an oral dosage unit comprising at least 2 g of an aliphatic amine polymer, at least 2 g of sevelamer or at least 0.5 g of a lanthanum salt, wherein the oral dosage unit is a tablet sachet, slurry, suspension or food formulation.

The methods of the present invention reduce the frequency of administration of phosphate binder to once daily, which will improve patient compliance and phosphate binding effectiveness.

### DETAILED DESCRIPTION OF THE INVENTION

Phosphate binders are currently administered with each meal (e.g., at least two or three times a day), leading to problems with patient compliance and thus the effectiveness of the therapy. The present invention discloses a once-per-day phosphate binder formulation that is substantially equivalent to the standard formulation requiring three times per day dosing for controlling serum phosphate. This once-per-day formulation is expected to improve patient compliance.

In one embodiment the present invention is a method for reducing serum phosphate in a subject in need thereof comprising administering once per day to said subject a phosphate binder, wherein the phosphate binder has a phosphate binding capacity of at least 52 mmole. Preferably the phosphate binder has a phosphate binding capacity of at least 78 mmole, at least 104 mmole, at least 130 mmole, at least 156 mmole, at least 182 mmole, or at least 269 mmole. More preferably the phosphate binder has a phosphate binding capacity in the range of 52 mmole to 269 mmole, 156 mmole to 182 mmole or 169 mmole to 174 mmole.

Phosphate binding capacity is defined herein as a measure of the in vitro ability of a phosphate binder to bind phosphate, monohydrogen phosphate or dihydrogen phosphate using the methods described in Rosenbaum et al. (Nephrol. Dial. Transplant. (1997) 12: 961-964, the entire contents of which are incorporated herein by reference).

In another embodiment the present invention is a method for reducing serum phosphate in a subject in need thereof, comprising administering once per day to said subject at least 2 g, preferably between 2 g and 10 g, between 3 g and 9 g, between 4 g and 8 g, between 6 g and 7 g, or between 6.5 g and 6.7 g of aliphatic amine polymer.

Amine polymers are characterized by a repeat unit that includes at least one amino group. Amino groups can be part of the polymer backbone (e.g., a polyalkyleneimine such as polyethyleneimine), pendant from the polymer backbone (e.g., polyallylamine), or both types of amino groups can exist within the same repeat unit and/or polymer. Amine polymers include aliphatic amine polymers and aromatic amine polymers.

An aliphatic amine polymer is obtained by polymerizing an aliphatic amine monomer. An aliphatic amine is saturated or unsaturated, straight-chained, branched or cyclic non-aromatic hydrocarbon having an amino substituent and optionally one or more additional substituents. An aliphatic amine monomer is an aliphatic amine comprising a polymerizable group such as an olefin. One example of a suitable aliphatic amine polymer is characterized by one or more repeat units of Structural Formula I: 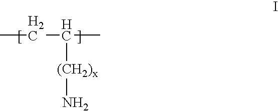
or a pharmaceutically acceptable salt thereof, where x is 0 or an integer between 1 and 4, preferably 1. The polymer represented by Structural Formula I is advantageously crosslinked by means of a multifunctional cross-linking agent.

Further examples of aliphatic amine polymers include polymers characterized by one or more repeat units set forth below: 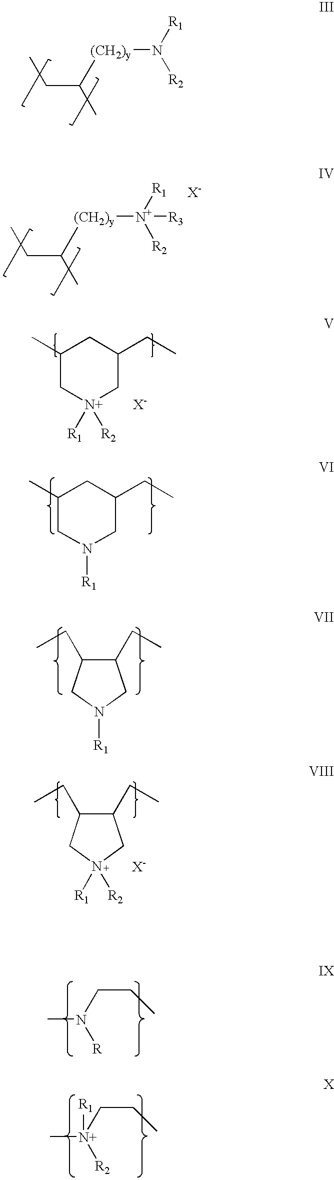
wherein y is an integer of zero, one or more (e.g., between about 1 and 10, 1 and 6, 1 and 4 or 1 and 3) and each R, R₁, R₂, and R₃, independently, is H or a substituted or unsubstituted alkyl group (e.g., having between 1 and 25, preferably between 1 and 5 carbon atoms, such as aminoalkyl having e.g., between 1 and 5 carbons atoms, inclusive, such as aminoethyl or poly(aminoethyl)) or substituted or unsubstituted aryl (e.g., phenyl) group, and each X⁻ is independently an exchangeable negatively charged counterion. Typically, R, R₁, R₂, and R₃ are each independently H or a substituted or unsubstituted alkyl group.

In one preferred polymer used in the invention, at least one of the R, R₁, R₂, or R₃ groups is a hydrogen atom. In a more preferred embodiment, each of these groups are hydrogen. In one embodiment, R, R₁, R₂, and R₃ are H and the polymer comprises repeat units characterized by Structural Formulas III, IV, V, VI, IX and/or X.

As an alkyl, or aryl group, R, R₁, R₂, or R₃ can carry one or more substituents. Suitable substituents include cationic groups, e.g., quaternary ammonium groups, or amine groups, e.g., primary, secondary or tertiary alkyl or aryl amines. Examples of other suitable substituents include hydroxy, alkoxy, carboxamide, sulfonamide, halogen, alkyl, aryl, hydrazine, guanadine, urea, poly(alkyleneimine), such as poly(ethyleneimine), and carboxylic acid esters.

A preferred polymer for use in the invention is polyallylamine, which is a polymer having repeat units from polymerized allyl amine monomers. The amine group of an allyl monomer can be unsubstituted or substituted with, for example, one or two C1-C10 straight chain or branched alkyl groups. The alkyl groups are optionally substituted with one or more hydroxyl, amine, halo, phenyl, amide or-nitrile groups. Preferably, the polyallylamine polymers of the present invention comprise repeat units represented by Structural Formula II:

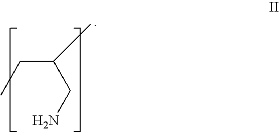

A polyallylamine can be a copolymer comprising repeat units from two or more different polymerized allyl monomers or with repeat units from one or more polymerized allyl monomers and repeat units from one or more polymerized non-allyl monomers. Examples of suitable non-allyl monomers include acrylamide monomers, acrylate monomers, maleic acid, malimide monomers, vinyl acylate monomers and alkyl substituted olefines. Preferably, however, the polyallylamines used in the present invention comprise repeat units solely from polymerized allyl amine monomers. More preferably, the polyallylamine polymers used in the present invention are homopolymers. Even more preferably, the polyallylamine polymers used in the present invention are homopolymers of repeat units represented by Structural Formula II or are crosslinked homopolymers thereof.

Amine polymers used in the invention are optionally protonated, and in one embodiment, include polymers in which less than 40%, less than 30%, less than 20% or less than 10% of the amine groups are protonated. In another embodiment 35% to 45% of the amines are protonated (e.g., approximately 40%), such as Renagel® which is commercially available from Genzyme Corporation.

An amine polymer can be a homopolymer or a copolymer of one or more amine-containing monomers or a copolymer of one or more amine-containing monomers in combination with one or more non-amine containing monomers. Copolymers that include one or more repeat units represented by the above Structural Formulas I-X, contain comonomers that are preferably inert and non-toxic. Examples of suitable non-amine-containing monomers include vinyl alcohol, acrylic acid, acrylamide, and vinylformamide.

Preferably, an aliphatic amine polymer is a homopolymer, such as a homopolyallylamine, homopolyvinylamine, homopolydiallylamine or polyethyleneamine. The word “amine,” as used herein, includes primary, secondary and tertiary amines, as well as ammonium groups such as trialkylammonium.

Aromatic amine polymers comprise an amine-containing aromatic moiety in one or more of the repeat units. An example of an aromatic amine polymer is poly(aminostyrene).

The preferred polymers employed in the invention are water-insoluble, non-absorbable, optionally cross-linked polyamines. Preferred polymers are aliphatic. Examples of preferred polymers include polyethyleneimine, polyallylamine, polyvinylamine and polydiallylamine polymers. The polymers can be homopolymers or copolymers, as discussed above, and can be substituted or unsubstituted. These and other polymers which can be used in the claimed invention have been disclosed in U.S. Pat. Nos. 5,487,888; 5,496,545; 5,607,669; 5,618,530; 5,624,963; 5,667,775; 5,679,717; 5,703,188; 5,702,696; 5,693,675; 5,900,475; 5,925,379; 6,083,497; 6,177,478; 6,083,495; 6,203,785; 6,423,754; 6,509,013; 6,556,407; 6,605,270; and 6,733,780 the contents of which are hereby incorporated herein by reference in their entireties. Polymers suitable for use in the invention are also disclosed in U.S. application Ser. No. 08/823,699 (now abandoned); Ser. No. 08/835,857 (now abandoned); Ser. No. 08/470,940 (now abandoned); Ser. No. 08/927,247 (now abandoned); Ser. Nos. 08/964,498; 09/691,429; 10/125,684; 10/158,207; 10/322,904; 10/441,157; and 10/766,638, the contents of which are incorporated herein by reference in their entireties.

Preferably, the polymer is rendered water-insoluble by cross-linking such as with a multifunctional cross-linking agent. The cross-linking agent is typically characterized by functional groups which react with the amino group of the monomer. Alternatively, the cross-linking agent can be characterized by two or more vinyl groups which undergo free radical polymerization with the amine monomer. The degree of polymerization in cross-linked polymers cannot generally be determined.

Examples of suitable multifunctional cross-linking agents include diacrylates and dimethylacrylates (e.g. ethylene glycol diacrylate, propylene glycol diacrylate, butylene glycol diacrylate, ethylene glycol dimethacrylate, propylene glycol dimethacrylate, butylene glycol dimethacrylate, polyethyleneglycol dimethacrylate and polyethyleneglycol diacrylate), methylene bisacrylamide, methylene bismethacrylamide, ethylene bisacrylamide, ethylene bismethacrylamide, ethylidene bisacrylamide, divinylbenzene, bisphenol A, dimethacrylate and bisphenol A diacrylate. The cross-linking agent can also include acryloyl chloride, epichlorohydrin, butanediol diglycidyl ether, ethanediol diglycidyl ether, succinyl dichloride, the diglycidal ether of bisphenol A, pyromellitic dianhydride, toluene diisocyanate, ethylene diamine and dimethyl succinate.

The level of cross-linking renders the polymers insoluble and substantially resistant to absorption and degradation, thereby limiting the activity of the polymer to the gastrointestinal tract, and reducing potential side-effects in the patient. The compositions thus tend to be non-systemic in activity. Typically, the cross-linking agent is present in an amount from about 0.5-35% or about 0.5-25% (such as from about 2.5-20% or about 1-10%) by weight, based upon total weight of monomer plus cross-linking agent.

In some cases the polymers are crosslinked after polymerization. One method of obtaining such crosslinking involves reaction of the polymer with difunctional crosslinkers, such as epichlorohydrin, succinyl dichloride, the diglycidyl ether of bisphenol A, pyromellitic dianhydride, toluence diisocyanate, and ethylenediamine. A typical example is the reaction of poly(ethyleneimine) with epichlorohydrin. In this example the epichlorohydrin (1 to 100 parts) is added to a solution containing polyethyleneimine (100 parts) and heated to promote reaction. Other methods of inducing crosslinking on already polymerized materials include, but are not limited to, exposure to ionizing radiation, ultraviolet radiation, electron beams, radicals, and pyrolysis.

Examples of preferred crosslinking agents include epichlorohydrin, 1,4 butanedioldiglycidyl ether, 1,2 ethanedioldiglycidyl ether, 1,3-dichloropropane, 1,2-dichloroethane, 1,3-dibromopropane, 1,2-dibromoethane, succinyl dichloride, dimethylsuccinate, toluene diisocyanate, acryloyl chloride, and pyromellitic dianhydride. Epichlorohydrin is a preferred crosslinking agent, because of its high availability and low cost. Epichlorohydrin is also advantageous because of its low molecular weight and hydrophilic nature, increasing the water-swellability and gel properties of the polyamine. Epichlorohydrin forms 2-hydroxypropyl crosslinking groups. In a preferred embodiment, the present invention is a polyallylamine polymer crosslinked with epichlorohydrin.

Typically, between about 9% and about 30% of the allylic nitrogen atoms are bonded to a crosslinking group, preferably between 15% and about 21%.

The polymers can also be further derivatized; examples include alkylated amine polymers, as described, for example, in U.S. Pat. Nos. 5,679,717, 5,607,669 and 5,618,530, the teachings of which are incorporated herein by reference in their entireties. Preferred alkylating agents include hydrophobic groups (such as aliphatic hydrophobic groups) and/or quaternary ammonium- or amine-substituted alkyl groups.

Non-cross-linked and cross-linked polyallylamine and polyvinylamine are generally known in the art and are commercially available. Methods for the manufacture of polyallylamine and polyvinylamine, and cross-linked derivatives thereof, are described in the above U.S. Patents. Patents by Harada et al., (U.S. Pat. Nos. 4,605,701 and 4,528,347), which are incorporated herein by reference in their entireties, also describe methods of manufacturing polyallylamine and cross-linked polyallylamine. A patent by Stutts et al., (U.S. Pat. No. 6,180,754) describes an additional method of manufacturing cross-linked polyallylamine.

In other embodiments, the polymer can be a homopolymer or copolymer of polybutenylamine, polylysine, or polyarginine. Alternatively, the polymer can be an aromatic polymer, such as an amine or ammonium-substituted polystyrene, (e.g., cholestyramine).

The molecular weight of polymers of the invention is not believed to be critical, provided that the molecular weight is large enough so that the polymer is non-absorbable by the gastrointestinal tract. Typically the molecular weight is at least 1000. For example the molecular can be from: about 1000 to about 5 million, about 1000 to about 3 million, about 1000 to about 2 million or about 1000 to about 1 million.

As described above, the polymer can be administered in the form of a salt. By “salt” it is meant that the nitrogen group in the repeat unit is protonated to create a positively charged nitrogen atom associated with a negatively charged counterion.

The anionic counterions can be selected to minimize adverse effects on the patient, as is more particularly described below. Examples of suitable counterions include organic ions, inorganic ions, or a combination thereof, such as halides (Cl⁻ and Br⁻), CH₃OSO₃⁻, HSO₄⁻, SO₄²⁻, HCO₃⁻, CO₃²⁻, acetate, lactate, succinate, propionate, oxalate, butyrate, ascorbate, citrate, dihydrogen citrate, tartrate, taurocholate, glycocholate, cholate, hydrogen citrate, maleate, benzoate, folate, an amino acid derivative, a nucleotide, a lipid, or a phospholipid. Preferred anions are Cl⁻, HCO₃⁻ and CO₃²⁻. The counterions can be the same as, or different from, each other. For example, the polymer can contain two or more different types of counterions.

A particularly preferred polymer is an epichlorohydrin cross-linked polyallylamine, such as sevelamer. In a preferred embodiment, the polyallylamine polymer is crosslinked with epichlorohydrin and between about 9% to about 30% (preferably about 15% to about 21%) of the allylic nitrogen atoms are bonded to a crosslinking group and the anion is chloride, carbonate or bicarbonate. More preferably, the polyallylamine polymer is a homopolymer. More preferably a polyallylamine polymer is a homopolymer comprising crosslinked repeat units represented by Structural Formula II.

In another preferred embodiment, the polyallylamine polymer used in the present invention is homopolyallyamine, preferably polyallylamine hydrochloride crosslinked with about 9.0-9.8% w/w epichlorohydrin, preferably 9.3-9.5%, and is the active chemical component of the drug known as sevelamer HCl, sold under the tradename RENAGEL. The structure is represented below: 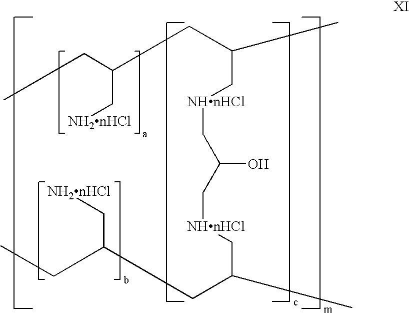
where:
the sum of a and b (the number of primary amine groups) is 9;
c (the number of crosslinking groups) is 1;
n (the fraction of protonated amines) is 0.4; and
m is a large number (to indicate extended polymer network).
   Typically, the amount of epichlorohydrin is measured as a percentage of the combined weight of polymer and crosslinking agent. In another preferred embodiment the polyallylamine polymer is sevelamer carbonate or sevelamer bicarbonate or a mixed carbonate and/or bicarbonate and chloride salt of sevelamer. Other examples of carbonate salts are disclosed in provisional U.S. Application Nos. 60/624,001 and 60/628,752, the entire contents of which are incorporated herein by reference.

The method of the present invention can also be used with other phosphate binders including pharmaceutically acceptable lanthanum, calcium, aluminum and iron salts, such as acetates, carbonates, oxides, hydroxides, citrates, alginates, and ketoacids. Calcium salts, including calcium carbonate, acetate (such as PhosLo® calcium acetate tablets), citrate, alginate, and ketoacids, have been utilized for phosphate binding. The tablets), citrate, alginate, and ketoacids, have been utilized for phosphate binding. The ingested calcium combines with phosphate to form insoluble calcium phosphate salts such as Ca₃(PO₄)₂, CaHPO₄, or Ca(H₂PO₄)₂. Aluminium-based phosphate binders, such as Amphojel® aluminium hydroxide gel, have also been used for treating hyperphosphatemia. These compounds complex with intestinal phosphate to form highly insoluble aluminium phosphate; the bound phosphate is unavailable for absorption by the patient. More recently iron and lanthanide salts have been used. The most commonly used lanthanide salt, lanthanum carbonate (Fosrenol®) behaves similarly to calcium carbonate.

As used herein, the term pharmaceutically acceptable salt refers to a salt of a compound to be administered prepared from pharmaceutically acceptable non-toxic acids including inorganic acids, organic acids, solvates, hydrates, or clathrates thereof. Examples of such inorganic acids are hydrochloric, hydrobromic, hydroiodic, nitric, sulfuric, and phosphoric. Appropriate organic acids may be selected, for example, from aliphatic, aromatic, carboxylic and sulfonic classes of organic acids, examples of which are formic, acetic, propionic, succinic, camphorsulfonic, citric, fumaric, gluconic, isethionic, lactic, malic, mucic, tartaric, para-toluenesulfonic, glycolic, glucuronic, maleic, furoic, glutamic, benzoic, anthranilic, salicylic, phenylacetic, mandelic, embonic (pamoic), methanesulfonic, ethanesulfonic, pantothenic, benzenesulfonic (besylate), stearic, sulfanilic, alginic, galacturonic, and the like.

In another embodiment the present invention is a method for reducing serum phosphate in a subject in need thereof, comprising administering once per day to said subject at least 0.5 g, preferably between at least 0.5 g and 10 g, between at least 0.5 g and 5 g, between at least 1 g and about 3 g, or between at least 1.5 g and about 2.25 g of a pharmaceutically acceptable lanthanum salt. In a preferred embodiment, the lanthanum salt is lanthanum carbonate.

The present invention also provides oral dosage units of phosphate binders that are particularly suitable for once-per-day administration. In one embodiment, the present invention is an oral dosage unit comprising at least 2 g, preferably between at least 2 g and 10 g, between at least 3 g and 9 g, between at least 4 g and 8 g, between at least 6 g and 7 g, or between at least 6.5 g and 6.7 g of the aliphatic amine polymer or a pharmaceutically acceptable salt thereof, wherein the oral dosage unit is a tablet, sachet, slurry, suspension or food formulation. In a preferred embodiment of the present invention the oral dosage unit is a sachet. Preferably the aliphatic amine polymer is a polyallylamine such as sevelamer.

In another embodiment the present invention is an oral dosage unit comprising at least 0.5 g, preferably between at least 0.5 g and 5 g, between at least 1 g and 3 g, or between at least 1.5 g and 2.25 g of lanthanum salt, wherein the oral dosage unit is a tablet, capsule, sachet, slurry, suspension or food formulation. In a preferred embodiment the oral dosage unit is a tablet.

Phosphate binders are advantageously administered in combination with a mucoadhesive. As used herein a mucoadhesive is a substance having the ability to adhere or to remain associated with a mucus tissue or membrane for extended periods of time. Examples of mucoadhesives include carboxymethyl and hydroxypropyl methyl cellulose, and other cellulose derivatives; tragacanth, caraya, locust bean and other synthetic and natural gums such as algin, chitosan, starches, pectins, and naturally-occurring resins, polyvinyl pyrrolidone, polyvinyl alcohol, and polyacrylic acid. More preferably the mucoadhesive is polyacrylic acid.

In one embodiment the phosphate binders of the present invention are administered before, during or after a meal. In a preferred embodiment the phosphate binder is administered before or after a meal. In a more preferred embodiment the phosphate binder is administered before a meal. The meal is preferably the largest meal of the day. As used herein, “before” or “after” a meal is typically within two hours, preferably within one hour, more preferably within thirty minutes, most preferably within ten minutes of commencing or finishing a meal, respectively.

The phosphate binder can be administered as multiple dosage units or preferably as a single dosage unit. As used herein a dosage unit may be a tablet, sachet, slurry, food formulation, troche, capsule, elixir, suspension, syrup, wafer, chewing gum or the like prepared by art recognized procedures. Preferably a dosage unit is a tablet, capsule, sachet, slurry, suspension or food formulation, more preferably the dosage unit is a tablet, slurry, suspension or food formulation, most preferably the dosage unit is a tablet or sachet. Typically, the desired dose of an aliphatic amine polymer is administered as multiple tablets or capsules, or a single dose of a sachet, slurry, food formulation, suspension or syrup.

In one example, the dosage unit is an oval, film coated, compressed tablet of Renagel containing either 800 mg or 400 mg of sevelamer hydrochloride on an anhydrous basis. The inactive ingredients are hypromellose, diacetylated monoglyceride, colloidal silicon dioxide, and stearic acid. In yet another embodiment, the dosage unit is a hard-gelatin capsule of Renagel containing 403 mg of sevelamer hydrochloride on an anhydrous basis. The inactive ingredients are colloidal silicon dioxide and stearic acid.

In a preferred embodiment, the dosage unit is a sachet comprising an aliphatic amine polymer, preferably polyallylamine, more preferably sevelamer hydrochloride.

In another preferred embodiment the dosage unit is a chewable tablet comprising lanthanum carbonate.

The phosphate binders of the present invention are preferably administered orally. The phosphate binders of the present invention can be administered to the subject alone or in a pharmaceutical composition, and optionally, one or more additional drugs. The pharmaceutical compositions of the invention preferably contain a pharmaceutically acceptable carrier or diluent suitable for rendering the compound or mixture administrable orally. The active ingredients may be admixed or compounded with a conventional, pharmaceutically acceptable carrier or diluent. It will be understood by those skilled in the art that any mode of administration, vehicle or carrier conventionally employed and which is inert with respect to the active agent may be utilized for preparing and administering the pharmaceutical compositions of the present invention. Illustrative of such methods, vehicles and carriers are those described, for example, in Remington's Pharmaceutical Sciences, 18th ed. (1990), the disclosure of which is incorporated herein by reference.

The formulations of the present invention for use in a subject comprise the agent, together with one or more acceptable carriers or diluents therefore and optionally other therapeutic ingredients. The carriers or diluents must be “acceptable” in the sense of being compatible with the other ingredients of the formulation and not deleterious to the recipient thereof. The formulations can conveniently be presented in unit dosage form and can be prepared by any of the methods well known in the art of pharmacy. All methods include the step of bringing into association the agent with the carrier or diluent which constitutes one or more accessory ingredients. In general, the formulations are prepared by uniformly and intimately bringing into association the agent with the carriers and then, if necessary, dividing the product into unit dosages thereof.

Those skilled in the art will be aware that the amounts of the various components of the compositions of the invention to be administered in accordance with the method of the invention to a subject will depend upon those factors noted above.

The compositions of the invention can be formulated as a tablet, sachet, slurry, food formulation, troche, capsule, elixir, suspension, syrup, wafer, chewing gum or lozenge. A syrup formulation will generally consist of a suspension or solution of the compound or salt in a liquid carrier, for example, ethanol, glycerine or water, with a flavoring or coloring agent. Where the composition is in the form of a tablet, one or more pharmaceutical carriers routinely used for preparing solid formulations can be employed. Examples of such carriers include magnesium stearate, starch, lactose and sucrose. Where the composition is in the form of a capsule, the use of routine encapsulation is generally suitable, for example, using the aforementioned carriers in a hard gelatin capsule shell. Where the composition is in the form of a soft gelatin shell capsule, pharmaceutical carriers routinely used for preparing dispersions or suspensions can be considered, for example, aqueous gums, celluloses, silicates or oils, and are incorporated in a soft gelatin capsule shell.

As used herein a subject is a mammal, preferably a human, but can also be an animal in need of veterinary treatment, such as a companion animal (e.g., dogs, cats, and the like), a farm animal (e.g., cows, sheep, pigs, horses, and the like) or a laboratory animal (e.g., rats, mice, guinea pigs, and the like).

### EXAMPLE 1

Equivalence of Once a Day and Three Times a Day Sevelamer Dosing.

Sevelamer hydrochloride, a metal free, nonabsorbed polymer is approved for controlling phosphorus in chronic kidney disease (CKD) patients on hemodialysis when dosed three times a day with meals.

The objective of this study was to evaluate the equivalency of once a day and three times a day sevelamer dosing.

After a 2 week sevelamer run-in period, 18 patients were randomized to either sevelamer dosed once a day with the largest meal for 4 weeks followed by standard three times per day dosing with meals for another 4 weeks; or sevelamer dosed three times per day with meals for 4 weeks followed by once a day dosing with the largest meal for another 4 weeks. Serum phosphorous, calcium corrected for albumin, calcium phosphorous product (Ca×P), albumin, intact parathyroid hormone (iPTH), total-cholesterol (total-C), low density lipoprotein cholesterol (LDL-C), high density lipoprotein cholesterol (HDL-C), and triglycerides were analyzed.

The mean age of patients studied was 64 yrs, 72% of the patients were male, and 61% were African-American. The average daily dose of sevelamer was 6.7 g. The total daily dosage of sevelamer was maintained constant when patients switched between once a day dosing and three times a day dosing.

Once a day sevelamer dosing was statistically equivalent to three times per day dosing at controlling serum P, Ca, Ca×P, albumin, total-C, LDL-C, HDL-C and triglycerides. Bioequivalence was not demonstrated for iPTH, likely due to high variablility and low sample size.

TABLE 1
Equivalency of once a day and three times a day sevelamer dosing

| | Three times a day | Every day |
|---|---|---|
| | (TID) | (QD) |
| Phosphorus (mg/dL)* | 4.6 ± 0.3 | 5.0 ± 0.3 |
| Calcium (mg/dL)* | 9.5 ± 0.2 | 9.4 ± 0.2 |
| Calcium-Phosphorus Product mg²/dL²)* | 44.0 ± 2.8 | 47.3 ± 2.7 |
| Albumin (gm/dL)* | 3.8 ± 0.1 | 3.8 ± 0.1 |
| iPTH (pg/mL)** | 227.0 | 226.8 |
| Total Cholesterol (mg/dL)* | 132.5 ± 7.7 | 135.0 ± 7.8 |
| LDL Cholesterol (mg/dL)* | 58.1 ± 6.0 | 60.5 ± 5.4 |
| HDL Cholesterol (mg/dL)* | 39.2 ± 2.4 | 39.8 ± 2.4 |
| Non-HDL Cholesterol (mg/dL)* | 90.4 ± 7.8 | 92.5 ± 7.8 |
| Triglycerides (mg/dL)* | 148.4 ± 22.1 | 144.3 ± 24.0 |

*90% CI for the ratio is within the interval (0.8, 1.25)
**iPTH is presented as median

Both once a day and three times per day sevelamer dosing were well tolerated. There were no serious adverse events related to the study medication.

In this study, sevelamer was effective when dosed once daily. This alternative prescribing schedule is expected to improve compliance and lead to more effective phosphorus management in the long-term.

While this invention has been particularly shown and described with references to preferred embodiments thereof, it will be understood by those skilled in the art that various changes in form and details may be made therein without departing from the scope of the invention encompassed by the appended claims.

1-51. (canceled)

52. A method of reducing serum phosphate in a subject in need thereof, comprising administering only once per day to said subject a pharmaceutical composition comprising an amine polymer or pharmaceutically acceptable salt thereof.

53. The method of claim 52 wherein said amine polymer is an aliphatic amine polymer.

54. The method of claim 53 wherein said the aliphatic amine polymer comprises one or more repeat units represented by a formula selected from the group consisting of: 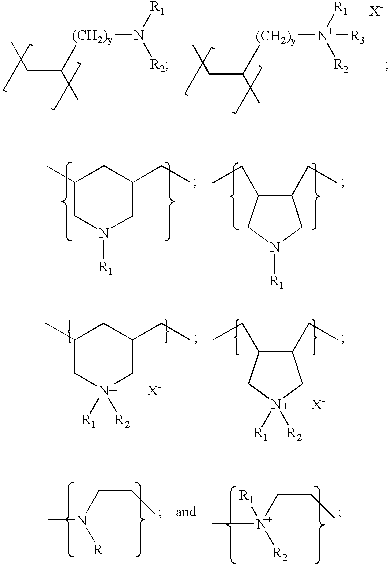
wherein:
y is an integer of zero, one or more;
R, R₁, R₂ and R₃, independently, represent H, a substituted or unsubstituted alkyl group or an aryl group; and
X⁻ is an exchangeable negatively charged counterion.

55. The method of claim 54 wherein said aliphatic amine polymer comprises a repeat unit represented by the following formula: 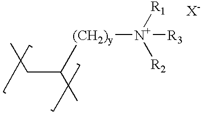
wherein:
y is an integer of zero, one or more;
R₁, R₂ and R₃, independently, represent H, a substituted or unsubstituted alkyl group or an aryl group; and
X⁻ is an exchangeable negatively charged counterion.

56. The method of claim 55 wherein y represents 1; R₁, R₂ and R₃ each represent hydrogen, and X⁻ represents chloride, carbonate, bicarbonate or a mixture of carbonate and bicarbonate.

57. The method of claim 56 wherein X⁻ represents chloride.

58. The method of claim 56 wherein X⁻ represents carbonate, or a mixture of carbonate and bicarbonate.

59. The method of claim 56 wherein said aliphatic amine polymer further comprises a second repeat unit represented by the formula: 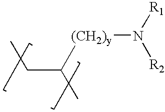
wherein y represents one, and
R₁ and R₂ each represent H.

60. The method of claim 58 wherein said aliphatic amine polymer further comprises a second repeat unit represented by the formula: 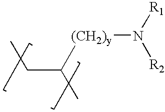
wherein y represents one, and
R₁ and R₂ each represent H.

61. The method of claim 52, wherein said pharmaceutical composition comprises at least 2 g of amine polymer.

62. The method of claim 58 wherein said pharmaceutical composition comprises between 3 g and 9 g of aliphatic amine polymer.

63. The method of claim 60 wherein said pharmaceutical composition comprises between 2 g and 10 g of aliphatic amine polymer.

64. The method of claim 60 wherein said pharmaceutical composition comprises between 3 g and 9 g of aliphatic amine polymer.

65. The method of claim 60 wherein said pharmaceutical composition comprises between 4 g and 8 g of aliphatic amine polymer.

66. The method of claim 56 wherein said aliphatic amine polymer is crosslinked.

67. The method of claim 60 wherein said aliphatic amine polymer is crosslinked.

68. The method of claim 64 wherein said aliphatic amine polymer is crosslinked.

69. The method of claim 52 wherein said pharmaceutical composition is administered within 2 hours before or after a meal.

70. The method of claim 56, wherein said pharmaceutical composition is administered within 2 hours before or after a meal.

71. The method of claim 67, wherein said pharmaceutical composition is administered within 2 hours before or after a meal.

72. The method of claim 71 wherein said meal is the largest meal of the day for said subject.

73. The method of claim 52 wherein said pharmaceutical composition is administered in the form of a sachet.

74. The method of claim 56 wherein said pharmaceutical composition is administered in the form of a sachet.

75. The method of claim 60 wherein said pharmaceutical composition is administered in the form of a sachet.

76. The method of claim 63 wherein said pharmaceutical composition is administered in the form of a sachet.

77. The method of claim 68 wherein said pharmaceutical composition is administered in the form of a sachet.

78. The method of claim 71 wherein said pharmaceutical composition is administered in the form of a sachet.

79. The method of claim 56 wherein said aliphatic amine polymer has a phosphate binding capacity of at least 52 mmole.

80. The method of claim 78 wherein said aliphatic amine polymer has a phosphate binding capacity of at least 52 mmole.

81. The method of claim 78 wherein said aliphatic amine polymer has a phosphate binding capacity of at least 104 mmole.

82. A method of treating hyperphosphatemia in a patient in need thereof comprising administering only once per day to said subject a pharmaceutical composition comprising an amine polymer.

83. The method of claim 82 wherein said amine polymer is an aliphatic amine polymer.

84. The method of claim 83 wherein said aliphatic amine polymer comprises a repeat unit represented by the following formula: 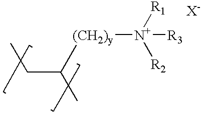
wherein:
y is an integer of zero, one or more;
R₁, R₂ and R₃, independently, represent H, a substituted or unsubstituted alkyl group or an aryl group; and
X⁻ is an exchangeable negatively charged counterion.

85. The method of claim 84 wherein X⁻ represents chloride.

86. The method of claim 84 wherein X⁻ represents carbonate or a mixture of carbonate and bicarbonate.

87. The method of claim 86 wherein said aliphatic amine polymer further comprises a second repeat unit represented by the formula: 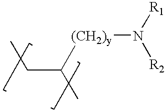
wherein y represents one, and
R₁ and R₂ each represent H.

88. The method of claim 87 wherein said pharmaceutical composition comprises between 2 g and 10 g of aliphatic amine polymer.

89. The method of claim 88 wherein said aliphatic amine polymer is crosslinked.

90. The method of claim 89, wherein said pharmaceutical composition is administered within 2 hours before or after a meal.

91. The method of claim 90 wherein said pharmaceutical composition in the form of a sachet.

92. The method of claim 91 wherein said aliphatic amine polymer has a phosphate binding capacity of at least 104 mmole.

93. The method of claim 86 wherein said pharmaceutical composition further comprises a pharmaceutically acceptable carrier or diluent.

94. The method of claim 91 wherein said pharmaceutical composition further comprises a pharmaceutically acceptable carrier or diluent.

95. A method of reducing serum phosphate in a subject in need thereof, consisting essentially of administering once per day to said subject a pharmaceutical composition comprising an amine polymer or pharmaceutically acceptable salt thereof.

96. The method of claim 95 wherein said amine polymer is an aliphatic amine polymer.

97. The method of claim 96 wherein said aliphatic amine polymer comprises a repeat unit represented by the following formula: 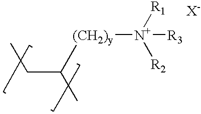
wherein:
y is an integer of zero, one or more;
R₁, R₂ and R₃, independently, represent H, a substituted or unsubstituted alkyl group or an aryl group; and
X⁻ is an exchangeable negatively charged counterion.

98. The method of claim 97 wherein: y represents 1; R₁, R₂ and R₃ each represent hydrogen, and X⁻ represents chloride, carbonate, bicarbonate or a mixture of carbonate and bicarbonate.

99. The method of claim 98 wherein X⁻ represents chloride.

100. The method of claim 98 wherein X⁻ represents carbonate, or a mixture of carbonate and bicarbonate.

101. The method of claim 100 wherein said aliphatic amine polymer further comprises a second repeat unit represented by the formula: 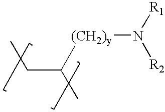
wherein y represents one, and
R₁ and R₂ each represent H.

102. The method of claim 101 wherein said pharmaceutical composition comprises between 3 g and 9 g of aliphatic amine polymer.

103. The method of claim 102 wherein said aliphatic amine polymer is crosslinked.

104. The method of claim 103, wherein said pharmaceutical composition is administered within 2 hours before or after a meal.

105. The method of claim 103 wherein said meal is the largest meal of the day for said subject.

106. The method of claim 103 wherein said pharmaceutical composition is administered in the form of a sachet.

107. The method of claim 105 wherein said pharmaceutical composition is administered in the form of a sachet.

108. The method of claim 101 wherein said aliphatic amine polymer has a phosphate binding capacity of at least 104 mmole.
